(19) United States
(12) Patent Application Publication
(10) Pub. No.: US 2013/0238060 A1
(43) Pub. Date: Sep. 12, 2013

(54) Phototherapy Device and Methods Thereof

(71) Applicant: Michael Olen NEVINS, Jackson, MI (US)

(72) Inventor: Michael Olen NEVINS, Jackson, MI (US)

(21) Appl. No.: 13/837,218

(22) Filed: Mar. 15, 2013

Related U.S. Application Data
(63) Continuation-in-part of application No. 12/274,322, filed on Nov. 19, 2008.
(60) Provisional application No. 61/003,604, filed on Nov. 19, 2007.

(51) Int. Cl. A61N 5/06; H05B 35/00

(52) U.S. Cl. CPC A61N 5/0613; H05B 35/00
USPC 607/90; 315/182

(57) ABSTRACT

A system for generating light flux including a lamp. The lamp having a plurality of light sources for illuminating an area. The light sources include a plurality of light-generating mechanisms at least one of which is different from the remainder. At least one of the light-generating mechanisms includes at least one of a fluorescent light source or an LED light source and at least one of the plurality of light sources having a different color temperature output than another one of the plurality of light sources. A light source controller electrically coupled with the light sources and electrically coupled to a biological spectrum setting to control the color temperature output of the light sources by applying a percentage allocation of each of the light sources. A switch electrically coupled between the lamp and a power supply and arranged to control supply of power to the lamp.

### RELATED APPLICATIONS

The present application is a continuation-in-part of U.S. application Ser. No. 12/274,322, filed Nov. 19, 2008, which is based on, and claims priority to prior provisional application entitled, “LED/fluorescent daylight tracking simulator and phototherapy device” having application serial number 61/003,604 filed on Nov. 19, 2007 and both of which are hereby incorporated herein by reference in its entirety.

### BACKGROUND

An example of an organ whose regulatory function is responsive to light sensed by the eyes is the pineal gland which secretes the hormone melatonin. The hormone is released during periods of darkness while production is abruptly halted when the eyes perceive bright light. Melatonin is distributed throughout the body via blood and cerebrospinal fluid and can effect the function of organs by which it is metabolized to thereby influence sleep cycles, feeding cycles, reproduction cycles and other biological rhythms. It has therefore been suggested that phototherapy may effectively be employed to correct a melatonin imbalance which may have resulted from, for example, shift work, jet lag or life in the Polar Regions, and thereby remedy the accompanying symptoms.

Millions of North Americans feel the effects of malillumination which causes poor work conditions and can result in less energy and productiveness. Poor lighting environments can cause increased depression and even result in more severe cases called Seasonal Affective Disorder (SAD). This problem increases more and more as the winter months bring shorter and shorter days. Sunlight starvation also effects millions more in the form of a milder form called Winter Blues.

Simulated full spectrum light is color corrected light that operates in the range of 400 to 800 nanometers. This light simulates the optical brilliance of outdoor light at noontime. This light can be measured by two numbers, the Color Rendering Index (CRI) and the Kelvin Temperature or (Degrees Kelvin). The secret to true color light and optically balanced light is how close you can get to the optics of natural light. The sun at noon has a natural color temperature of 100 CRI and between 5000 and 5500 degrees Kelvin. Both CRI and Kelvin are important for the simulation sunlight.

When light is simulated that matches the optical brilliance of sunlight pupils in one's eyes become smaller. This response generates clearer vision and higher perception. The results are lower glare and eye fatigue. When Lux intensity is combined with high CRI and balanced Kelvin temperature, quality light is obtained that not only matches the optical brilliance of the sun, but reduces levels of melatonin and the stress hormone, cortisol. Full spectrum light is not blue light or daylight color. It is clear, brilliant, white light and simulates the exact color of sunlight at noon. Many people currently progress through life missing sunlight because of the enormous amounts of time that are spent indoors.

Melatonin is a hormone that is believed to have a sleep inducing effect in humans. Melatonin is released by the pineal gland of humans, and the levels vary in a twenty-four hour cycle. Melatonin is believed involved in the circadian rhythm cycle of humans. It is believed that melatonin production is inhibited by light, and in particular blue light of about 460 nm to 480 nm suppresses melatonin production.

Melanopsin is a photopigment that absorbs light at a peak sensitivity of about 480 nm, which is in the blue color range. Melanopsin is found in ganglion cells of the retina and is believed to be involved in circadian rhythm regulation.

Many light sources contain a blue portion of the light spectrum, which corresponds to the sensitivity of the melanopsin containing ganglion cells within the eye of some mammals and humans. Stimulation of melanopsin containing ganglion cells at night may contribute to circadian rhythm and hormonal disruption.

Cortisol is a hormone produced by the adrenal gland of humans. Adrenal glands release cortisol in response to stress, and cortisol is involved in glucogenesis to increase blood sugar levels, suppression of the immune system and involved in metabolism of fats, proteins and carbohydrates. Cortisol awakening response is an increase in cortisol levels shortly after awakening for some humans. It is believed that cortisol eventually peaks shortly after awakening and then eventually decreases during the course of the day. During awakening, it is believed that exposure of humans to short wavelength light, for example 470 nm, increases the cortisol awakening response. Subsequently, exposure of humans to bright light during the course of the day has been shown to suppress cortisol levels in humans.

### DESCRIPTION OF THE DRAWINGS

One or more embodiments are illustrated by way of example, and not by limitation, in the figures of the accompanying drawings, wherein elements having the same reference numeral designations represent like elements throughout and wherein:

FIG. 1 is a side view of a lamp according to an embodiment;

FIG. 2 is a high level functional block diagram of a lamp according to an embodiment;

FIG. 3 is a high-level functional block diagram of a controller according to an embodiment;

FIG. 4 is a high level process flow diagram of a light controller usable in conjunction with an embodiment;

FIG. 5 is a side view of a lamp according to another embodiment;

FIG. 6 is a perspective view of a light box according to an embodiment;

FIG. 7 is a front view of a light window according to an embodiment;

FIG. 8 is a perspective view of a light tile according to an embodiment;

FIG. 9 is a perspective view of a room incorporating a lighting system according to an embodiment; and

FIG. 10 is a perspective view of a room incorporating a lighting system according to another embodiment.

### DETAILED DESCRIPTION

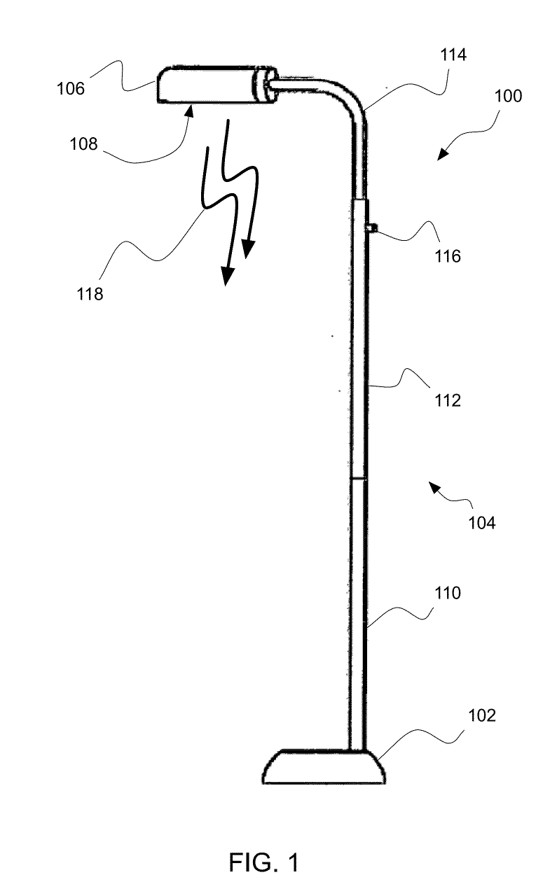

FIG. 1 depicts a freestanding lighting system 100 arranged to provide phototherapy and/or daylight tracking simulation according to one or more embodiments. Freestanding lighting system 100 may be placed on a floor surface and comprises a base support 102 which provides a stable support platform for the lighting system, a vertically extending connection member 104, a light source holder 106, and a light source 108. Light source 108 is configured to generate a photo-therapeutic flux (or luminance) from a florescent or LED-based light source. Lighting system 100 is arranged to selectively provide color changes as well as luminance intensity changes, i.e. locks intensity changes, based on a time schedule or specific phototherapy setting through the use of digital or analog controls, and/or computer programming or other control devices.

Vertically extending connection member 104 is cooperatively coupled with light source holder 106 at one and is cooperatively coupled with a support 102 at a distal end thereof. Connection member 104, as depicted in FIG. 1, comprises a first segment 110 connected at one end to base support 102, a second segment 112 connected to the first segment, and the third segment 114 connected at one end to the second segment and at the other end to light source holder 106. As depicted, third segment 114 comprises a curvilinear portion to change the direction of the segment from substantially vertical to horizontal.

Second segment 112 comprises a switch 116 for controlling operation of lighting system 100. In at least some embodiments, switch 116 may be positioned in another segment of the connection member 104, as part of base support 102, as part of light source holder 106, or remotely located from lighting system 100.

Light source 108 is positioned within light source folder 106 and, in operation, generates illumination (generally indicated by arrows identified by reference numeral 118). Light source 108 comprises a light-generating mechanism selected from at least one of a fluorescent lamp or a light emitting diode (LED) lamp. In at least some embodiments, light source 108 may comprise more than one lamp or light-generating mechanism. In at least some embodiments, light source 108 comprises either a fluorescent lamp or an LED lamp exclusive of another type of lamp, e.g., incandescent lamp or light source.

In at least some embodiments, one or more of a support 102 connection member 104, or light source holder 106 may be comprised of a metallic material. In at least some embodiments, the third segment 114 may comprise at least a portion of a flexible material enabling bending of light source holder with respect to the vertical extension of connection member 104.

In at least some embodiments, switch 116 is electrically coupled with light source 108 via wiring extending within or along third segment 114 of connection number 104. In at least some embodiments, lighting system 100 comprises an integrated power supply, e.g., a battery, or is configured to receive power from a power supply source, e.g., line or mains power.

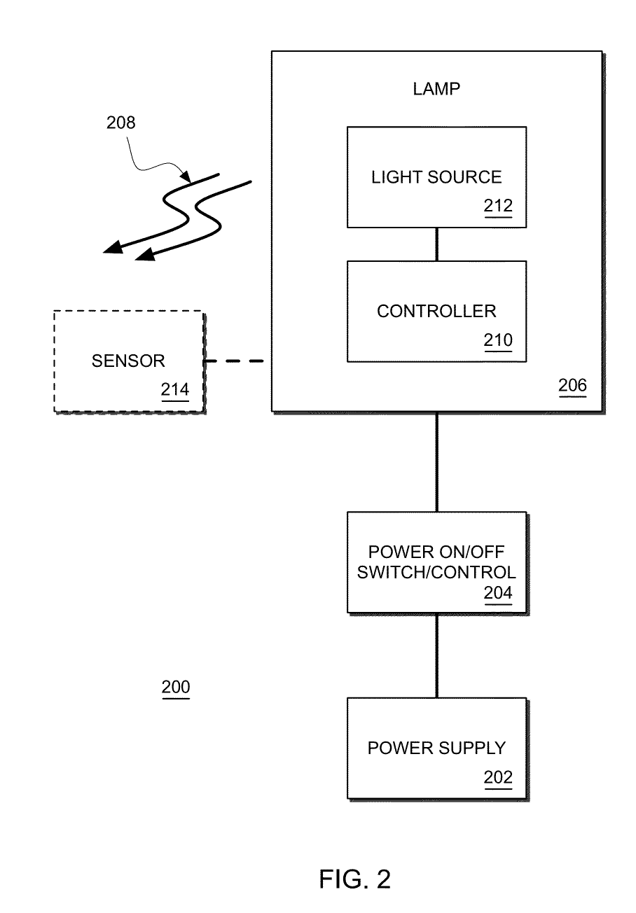

FIG. 2 depicts a high-level functional block diagram of a lighting system 200 (similar to lighting system 100 (FIG. 1)) according to an embodiment. Lighting system 200 comprises a power supply 202, which in some embodiments may alternatively be a power source, electrically coupled with a power on/off switch/control 204 for controlling the transmission of electrical power from power supply 202 to a lamp 206.

In at least some embodiments, switch/control 204 may be a switch, e.g., switch 116 (FIG. 1). Switch/control 204 may be configured in the form of an appropriate switch device for turning the lamp 206 on and off. For example, switch/control 204 may be a knob or dial rotatable in one direction to turn the lamp on, e.g. clockwise, and rotatable in the other direction to turn the lamp off, e.g. counterclockwise.

Switch/control 204 may alternatively be configured as a one and/or two push button control and may be used alternately or simultaneously. One push button operation may be effected by configuring switch/control 204 with one button, and pressing switch/control 204 button briefly, e.g., below a predetermined period of time, to switch the lamp 206 on or off. By pressing switch/control 204 button longer, e.g., above the predetermined period of time, the lamp 206 generates illumination 208 according to a different spectral output, e.g., warmer or cooler color output. The last spectral output may be stored in the lamp 206 when the lamp is switched off, and may be retrieved when the lamp is switched on.

In at least some embodiments, lamp 206 may be a light source holder, e.g., light source holder 106 (FIG. 1). Lamp 206 is electrically coupled with switch/control 204.

Lamp 206 comprises a light source controller 210 cooperatively coupled with a light source 212. In at least some embodiments, light source 212 is either a fluorescent light source or a light emitting diode (LED) light source. In at least some embodiments, light source 212 comprises at least two light sources where one of the light sources is a fluorescent light source and the other is an LED light source. In at least some other embodiments, light source 212 comprises at least one incandescent light source and at least one light source selected from a group comprising at least a fluorescent light source or an LED light source.

Light source controller 210 is arranged to control the spectrum output of light source 212. In at least some embodiments in which light source 212 comprises more than a single light source, light source controller 210 is arranged to control each light source individually or according to one or more groupings of light sources.

According to a multi-light source embodiment, light source 212 comprises a heterogeneous set of light sources in which each light source has a different color temperature output. For example, a first light source may have a correlated color temperature (CCT) of 8,000 Kelvin (K) whereas a second light source may have a CCT of 3,000 K. In some other embodiments, a first light source may have a CCT of 1,800 K, whereas a second light source may have a CCT of 3,000K. In accordance with such a heterogeneous multi-light source embodiment, controller 210 is arranged to vary the color temperature output of the combined light sources as light source 212 by varying the brightness of the individual light sources. For example, in order to achieve a first color temperature output level, controller 210 may cause the first light source brightness level to be set to output at 50% of the maximum output level of the light source and cause the second light source brightness level to be set to output at 75% of the maximum output level of the light source resulting in a color temperature output of light source 212 tending more toward the second light source color temperature, i.e., 3,000K. That is, a blending of the spectrum output of the individual light sources may be generated.

In another illustrative example, the first light source brightness level is set to output at 75% of the maximum output level of the light source and the second light source brightness level is set to output at 50% of the maximum output level of the light source resulting in a color temperature output of light source 212 tending more toward the first light source color temperature, i.e., 1,800K.

Color temperatures over 5,000 K are associated with a blueish hue and include light having wavelength of about 480 nm as well as other wavelengths. Color temperatures of 3,000 K or less are associated with a yellow or red hue and include light having wavelength of about 580 nm as well as other wavelengths.

In at least some embodiments, different numbers of light sources and different combinations of light sources having specific color temperature output may be combined to form light source 212. In at least one embodiment, a set of three heterogeneous light sources may be used in which a first light source color temperature is 10,000 K, a second light source color temperature is 3,500 K, and a third light source color temperature is 5,000 K. In at least one embodiment, a set of three heterogeneous light sources may be used in which the first light source color temperature is 1,800 K, a second light source color temperature is 3,500 K and a third light source color temperature is 12,000 K. Varying the brightness of the individual light sources enables lamp 206 to output different color temperature outputs .

In at least some embodiments, light source controller 210 adjusts the brightness of the individual light sources comprising light source 212 in order to obtain a particular color temperature output. The particular color temperature output by light source 212 may be monitored through the use of sensor 214. In at least some embodiments, a user may cause light source controller 210 to vary the color temperature output by manipulating switch/control 204. In at least some further embodiments, light source controller 210 is arranged to apply a particular percentage allocation to each of the light sources while varying the illumination intensity of the light sources at a constant level.

In at least some embodiments, a phosphor blend using multiple bands, e.g., from four to ten bands, is used in the light source to produce a desired blend that produces a balanced spectrum, as well as operate near the 580 nm peak of the scotopic curve. In some other embodiments, a phosphor blend is used in a light source to produce a spectrum that operates in a range greater than 480 nm of the scoptic curve.

In at least some embodiments, controller 210 is a discrete integrated circuit or set of integrated circuits configured to control light source 212 according to an embodiment. In at least some other embodiments, controller 210 is a processor or application specific integrated circuit (ASIC) configured to control light source 212 according to an embodiment.

In at least some embodiments, lighting system 200 also comprises a sensor 214 such as a light sensor configured to detect a frequency of the illumination 208 generated by light source 212. For example, sensor 214 may comprise a sensor to detect the color temperature output of light source 212. In at least some other embodiments, sensor 214 is a position determination system such as a global positioning satellite (GPS) system receiver arranged to determine one or both of a geographic location of lighting system 200 or a current date and/or time.

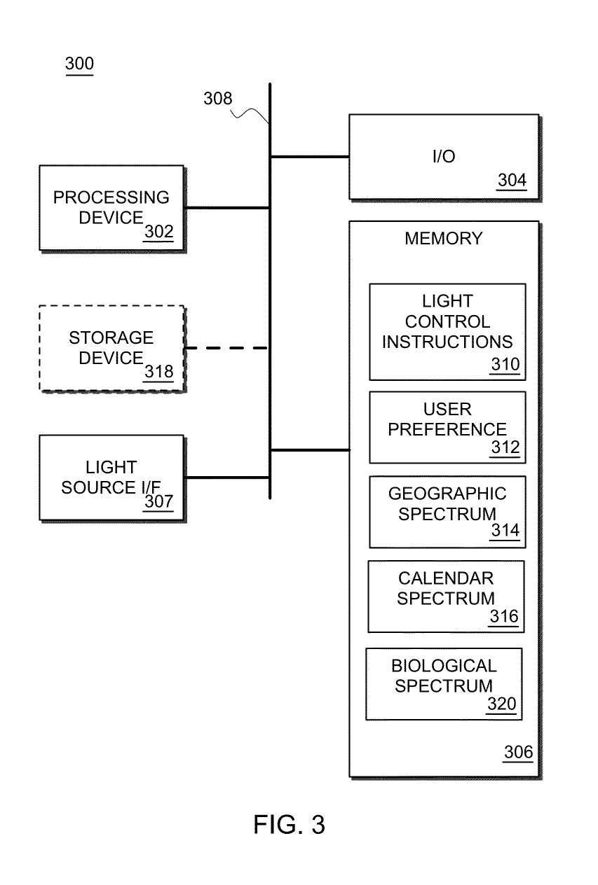

FIG. 3 depicts a high-level functional block diagram of a controller 300 according to an embodiment in conjunction with which an embodiment of the present invention may be executed to great advantage. Controller 300 comprises a processing device 302 (alternatively referred to as a processor), an input/output (I/O) device 304, a memory 306, and a light source interface (I/F) device 307 each communicatively coupled via a bus 308 or other interconnection communication mechanism.

In at least some embodiments, processing device 302 may be a controller and/or and application-specific integrated circuit (ASIC) configured to execute a set of instructions such as those embodied by an embodiment.

Memory 306 (also referred to as a computer-readable medium) may comprise a random access memory (RAM) or other dynamic storage device, coupled to the bus 308 for storing data and/or instructions to be executed by processing device 302, e.g., light control instructions 310, user preference(s) 312, geographic spectrum setting 314, calendar spectrum setting 316, or biological spectrum setting 320. Memory 306 also may be used for storing temporary variables or other intermediate information during execution of instructions to be executed by processing device 302. Memory 306 may also comprise a read only memory (ROM) or other static storage device coupled to the bus 308 for storing static information and instructions for the processing device 302.

A storage device (optional dashed line box 318), such as a magnetic, optical, electromagnetic, or holographic disk or other storage medium, may also be provided and coupled to the bus 308 for storing data and/or instructions.

In at least some embodiments, light control instructions 310 comprise a set of executable instructions which, when executed by processing device 302, cause the processing device to control a light source, e.g., light source 212 (FIG. 2).

I/O device 304 may comprise an input device, an output device and/or a combined input/output device for enabling user interaction. An input device may comprise, for example, a keyboard, keypad, mouse, trackball, trackpad, and/or cursor direction keys for communicating information and commands to processing device 302. An output device may comprise, for example, a display, a printer, a voice synthesizer, etc. for communicating information to a user. In at least some embodiments, I/O device 304 may comprise a serial and/or parallel connection mechanism for enabling the transfer of one or more of files and/or commands, e.g., an Ethernet or other type network connection.

In at least some embodiments, I/O device 304 is cooperatively coupled with sensor 214 in order to receive a signal representative of a color temperature output of light source 307. In at least some embodiments, I/O device 304 is cooperatively coupled with sensor 214 in order to receive a geographic location or a current date and/or time.

Light source I/F 307 comprises an electrical, optical, and/or electro-optical interface between controller 210 and light source 212 (FIG. 2). Light source I/F 307 connects controller 300 to a light source, e.g., light source 212, and enables the controller to control the color temperature output of the light source. For example, controller 300 via light source I/F 307 is able to turn on and off the light source and/or modify the output characteristics of the light source responsive to execution of light control instructions 310.

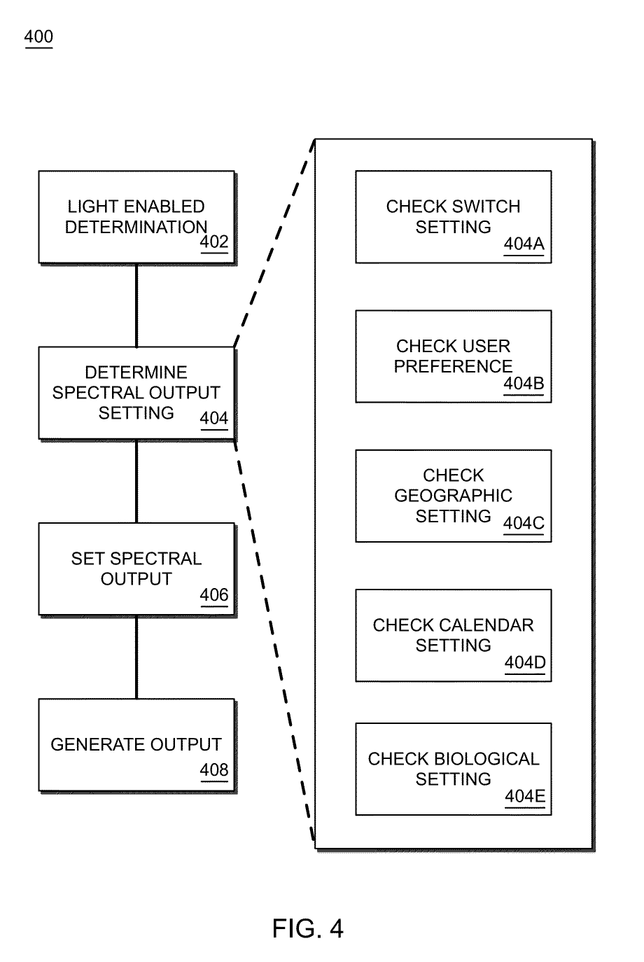

FIG. 4 depicts a high-level process flow diagram of at least a portion 400 of a method, e.g., execution of light control instructions 310 (FIG. 3) by processing device 302, according to an embodiment. The process flow begins at light enable determination functionality 402 wherein execution of light control instructions 310 by processing device 302 causes controller 300 to determine whether lighting system 200, e.g., via receipt of input from switch/control 204 (FIG. 2) or via another input device connected to I/O device 304, is turned on. In at least some embodiments, light enabled determination 402 may be eliminated and the receipt of power from power supply 202 (FIG. 2), either with or without switch/control 204 as appropriate, provides the functionality.

The flow then proceeds to determine spectral output setting functionality 404. During execution of functionality 404, lighting system 200 determines the color temperature output to be generated by light source 212 (FIG. 2). The determination may comprise one or more of reading a value from a memory location, e.g., user preference 312 of memory 306 (FIG. 3), or reading the position of switch/control 204.

In at least some embodiments, one or more of additional functionalities, i.e., check switch setting 404A, check user preference 404B, check geographic setting 404C, or check calendar setting 404D, may be executed in order to determine the spectral output setting.

Check switch setting functionality 404A causes processing device 302 to determine the position of switch/control 204 or another switch/control attached to lighting system 200 in order to determine the color temperature output desired.

Check user preference functionality 404B causes processing device 302 to read the value stored in user preference 312 of memory 306 (FIG. 3) in order to determine the color temperature output desired.

Check geographic setting functionality 404C causes processing device 302 to read the value stored in geographic spectrum setting 314 of memory 306 (FIG. 3) in order to determine the color temperature output desired. In at least some embodiments, geographic spectrum setting 314 may specify a particular color temperature output for each of one or more geographic locations, i.e., a different spectrum output may be specified for a different location. In at least some embodiments, check geographic setting functionality 404C may compare a stored geographic location with a determined current geographic location to determine whether the spectrum setting should be used. For example, the current geographic location may be determined with reference to an internal position-determining mechanism, a user-supplied geographic location, or via a geographic location determined by an external device such as sensor 214, e.g., a GPS-type or broadcast signal such as LORAN.

Check calendar setting functionality 404D causes processing device 302 to read the value stored in calendar spectrum setting 316 of memory 306 (FIG. 3) in order to determine the color temperature output desired. Calendar spectrum setting 316, in some embodiments, may further specify a period of time (either date or time of day) during which a particular color temperature output setting is valid. In at least some embodiments, calendar spectrum setting 316 may specify a particular color temperature output for each of one or more portions of a day, i.e., a different color temperature output may be specified for a different period of a given day. In at least some embodiments, check calendar setting functionality 404D may compare a stored date value with a determined current date to determine whether the color temperature output setting should be used. For example, the current date or time may be determined with reference to an internal clock or timer, a user-supplied date or time, or via a date or time determined by an external device such as sensor 214, e.g., a GPS-type or broadcast atomic signal.

Check biological spectrum setting functionality 404E causes processing device 302 to read the value stored in biological spectrum setting 320 of memory 306 (FIG. 3) in order to determine the desired color temperature output. In some embodiments, the value of the biological stored spectrum setting includes a color temperature output of approximately 1,800 K. In this manner, the biological spectrum setting 320 is set to reduce melatonin production and/or prevent absorption of light by melanopsin in humans.

In some other embodiments, the value of the biological spectrum setting 320 includes a color temperature output of approximately 5,000 K. In this manner, the biological spectrum setting 320 is set to increase the cortisol awakening response in humans during awakening. In some other embodiments, the biological spectrum setting 320 includes a color temperature output of approximately 12,000 K to suppress cortisol levels in humans during the daytime.

Biological spectrum setting 320, in some embodiments, may further specify a period of time during which a particular color temperature output is valid. In at least some embodiments, biological spectrum setting 320 may specify a particular color temperature output for each of one or more portions of a day, i.e., a different color temperature output may be specified for a different period of a given day.

In at least some embodiments, check biological spectrum setting 404E may compare a stored date value with a determined current date to determine whether the color temperature output should be used. For example, the current date or time may be determined with reference to an internal clock or timer, a user-supplied date or time, or via a date or time determined by an external device such as sensor 214, e.g., a GPS-type or broadcast atomic signal.

In at least one embodiment, the biological spectrum setting 320 is configured by a user. A user seeking to avoid exposure of blue light during sleep periods configures the value of the biological setting 320 to include a color temperature output of less than 5,000 K. In some embodiments, the user configures the biological setting 320 to include a desired color temperature output at a particular time of day and/or length of time.

In some embodiments, the user configures the biological spectrum setting 320 to include a color temperature output having a first value at one point in time, and a second value at a second point of time. In some embodiments, the user configures the biological spectrum setting 320 to include a first color temperature output less than 5,000 K during the night time, and a second color temperature output greater than 5,000 K during the morning and/or day time. The user's configuration of the biological spectrum setting 320 aids in the reduction of melatonin production in humans, prevention of absorption of light by melanopsin in humans, increase of the cortisol awakening response in humans, and/or suppression of cortisol levels in humans.

In at least some embodiments, user preference 312 also stores priority information specifying which particular setting, if more than one are present, takes priority over the other settings. For example, user preference 312 may indicate that if the date meets a predetermined threshold value, then the switch/control 204 may be used as the preferred color temperature output setting. On the other hand, if the geographic location of the lighting system 200 is within a predetermined distance of the geographic setting, then the calendar spectrum setting 314 may be used as the preferred color temperature output setting.

After determining the color temperature output setting to be used, the flow proceeds to set output functionality 406 wherein execution of the instructions causes processing device 302 to transmit the determined output setting to light source I/F 307. The flow then proceeds to generate output functionality 408 wherein processing device 302 causes light source I/F 307 to cause light source 212 to generate illumination having the determined color temperature output setting.

In at least some embodiments, functionality 406 and 408 may be combined into a single functionality performing the transmission of the color temperature output setting and activation of light source 212.

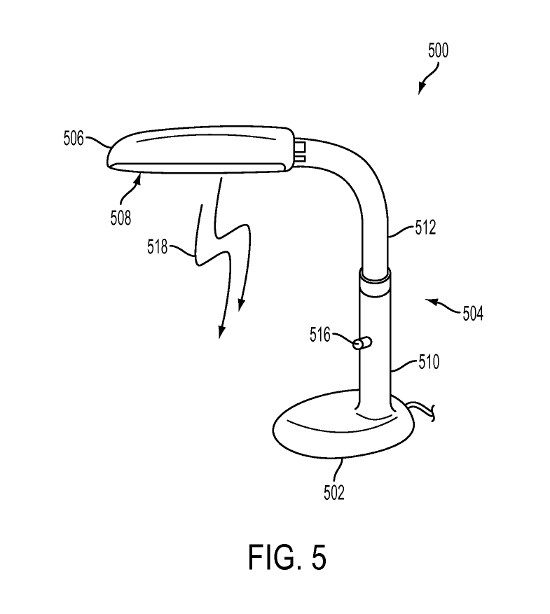

FIG. 5 depicts a side view of a lamp 500 according to another embodiment for a desk or task-based lamp. Similar to lamp 100, lamp 500 comprises a base support 502, a vertically extending connection member 505, a light source holder 506, and a light source 508. Connection member 505 comprises a segment 510 extending generally vertically and connected with a curved segment 512 forming an angle enabling illumination of a surface below lamp 500. Lamp 500 also comprises a switch/control 516 similar to switch/control 204 (FIG. 2). In operation, light source 508 generates and transmits illumination 518. Lamp 500 comprises a light control system similar to the light control system 300 (FIG. 3).

In at least some embodiments, curved segment 512 of lamp 500 is flexible enabling a user to modify the amount of curvature of the segment.

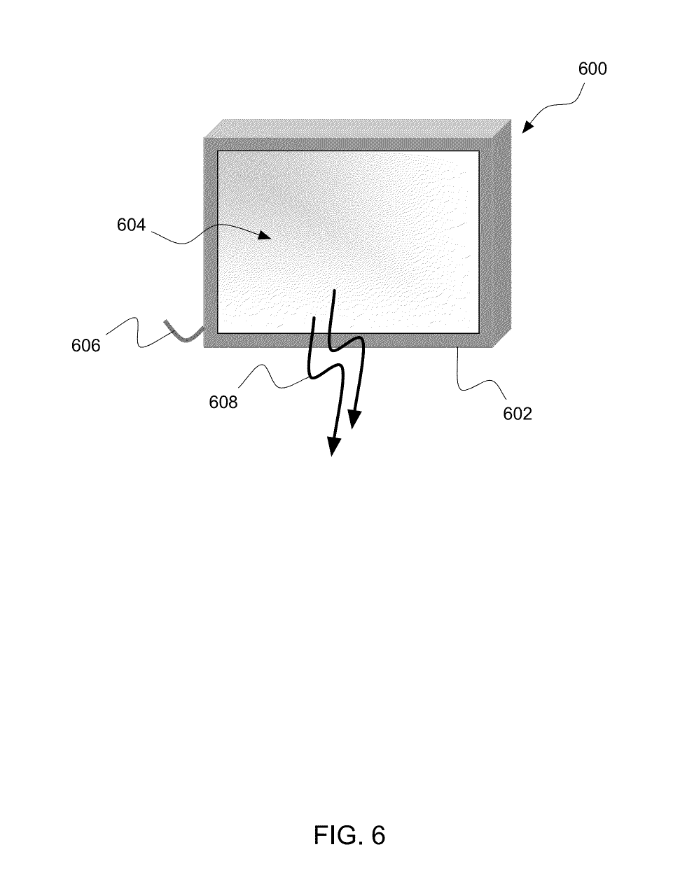

FIG. 6 depicts a perspective view of a light box 600 according to an embodiment. Light box 600 comprises a generally parallelepiped box 602 having a relatively large front face in comparison to the sides, top, and bottom. In at least some embodiments, box 602 may be different shapes and sizes without departing from the spirit and scope of the present embodiments.

The front face of box 602 comprises a light source holder 604. Light source holder 604 comprises a light source similar to light source 212 (FIG. 2). Box 600 comprises a power cord 606 for connecting the box to a power supply. In at least some embodiments, box 600 excludes the power cord 606 and relies on a stored power source such as a battery to power the box and the illumination 608 generation.

In operation, light source holder 604 generates and transmits illumination 608. Lamp 600 comprises a light control system similar to the light control system 300 (FIG. 3).

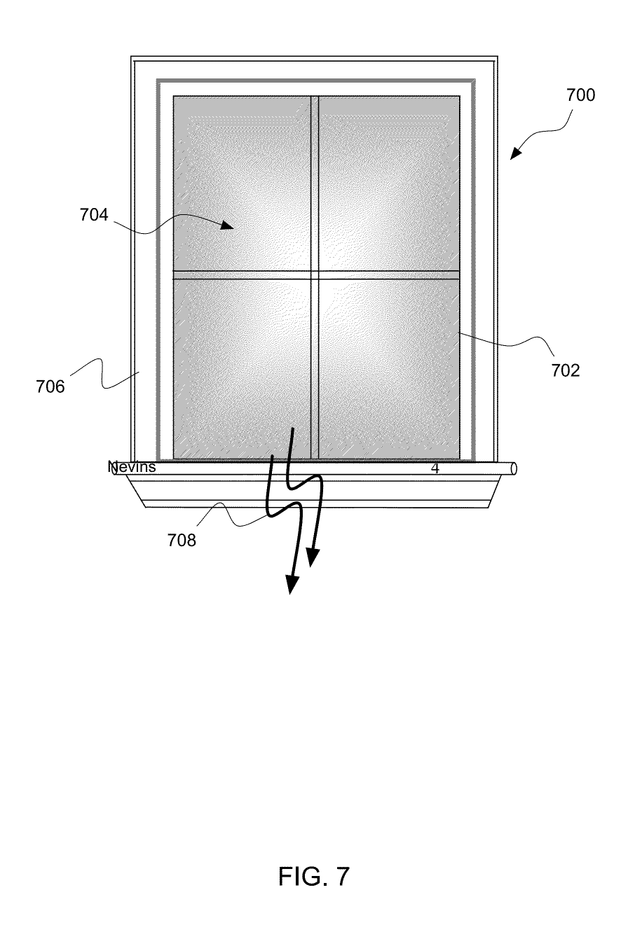

FIG. 7 depicts a front view of a light window 700 according to an embodiment. In operation, light window 700 may be used in place of or in addition to a nominal window allowing light to pass through. Light window 700 comprises a generally rectangular panel 702 comprising a light source holder 704. Light source holder 704 comprises a light source similar to light source 212 (FIG. 2).

Light window 700 also comprises a window frame 706 configured to replicate a normal window frame in use. In at least some embodiments, window frame 706 may be used to mount light window 700 on a wall or other vertical surface. In at least some other embodiments, window frame 706 may be a different size, shape, and/or configuration as appropriate for a particular location. For example, window frame 706 may be square, elliptical, circular, or otherwise shaped.

In operation, light source holder 704 generates and transmits illumination 708. Light window 700 comprises a light control system similar to the light control system 300 (FIG. 3).

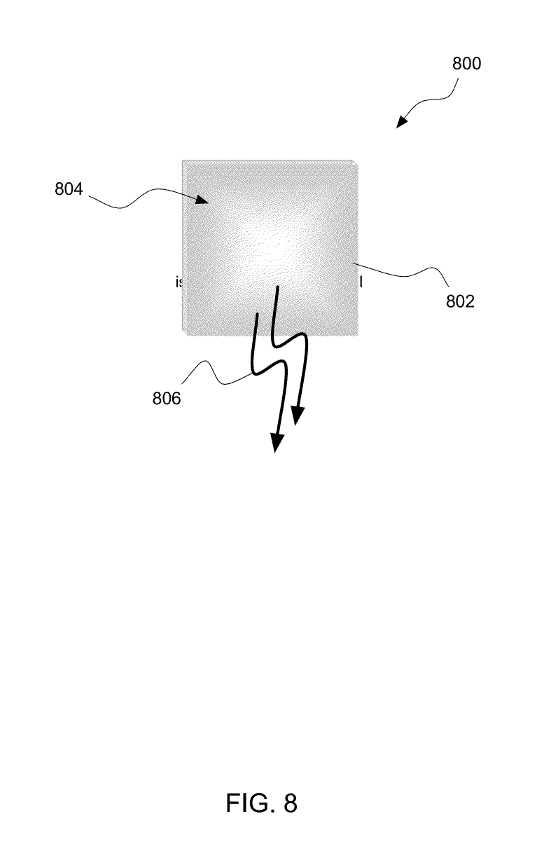

FIG. 8 depicts a perspective view of a light tile 800 according to an embodiment. In operation, light tile 800 may be used in place of or in addition to a nominal tile, e.g., as used in a home or office setting. Light tile 800 comprises a generally rectangular panel 802 comprising a light source holder 804. Light source holder 804 comprises a light source similar to light source 212 (FIG. 2).

In at least some other embodiments, light tile 800 may be different shapes and sizes without departing from the spirit and scope of the present embodiments. In at least one embodiment, light tile 800 is sized to fit within a user's briefcase and be transportable by a user. For example, in some embodiments, the light tile may be six, eight, ten, or at least twelve inches along at least one dimension.

In operation, light source holder 804 generates and transmits illumination 806. Light tile 800 comprises a light control system similar to the light control system 300 (FIG. 3). Light tile 800 may comprise a battery or other power source enabling the tile to be self-sufficient power-wise for a time period.

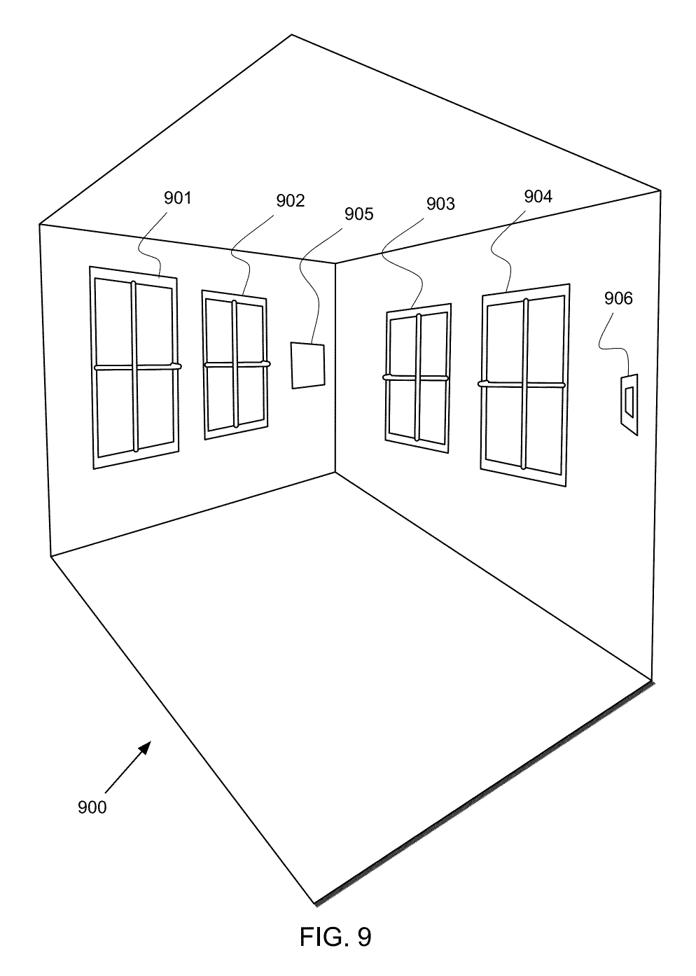

FIG. 9 depicts a perspective view of a room 900 incorporating a lighting system according to an embodiment. Room 900 comprises a set of light sources 901-904 constructed to appear as individual windows, e.g., similar in style to light window 700 (FIG. 7). Light sources 901-904 are cooperatively coupled with a light source controller 905 similar to controller 210 (FIG. 2). In at least some embodiments, light source controller 905 is identical to controller 210 and comprises a wired and/or wireless interface for communicating with light sources 901-904. Controller 905 is cooperatively coupled, e.g., via wired and/or wireless connection, with a switch/control 906 similar to switch/control 204 (FIG. 2). In at least some embodiments, switch/control 906 is identical to switch/control 204. In accordance with the FIG. 9 embodiment, a user in room 900 is able to adjust the spectrum output from light sources 901-904 via manipulation of switch/control 906 as is described above.

In at least some embodiments, controller 905 is electrically connected with a power supply such as a mains or line power supply. In at least some embodiments, light sources 901-904 are electrically connected with the power supply. In at least some embodiments, light sources 901-904 are electrically connected with controller 905 in order to receive power.

In at least some embodiments, light sources 901-904 each comprise an integrated individual light source controller and the individual light source controllers communicate, e.g., either wired and/or wirelessly, with each other and with switch/control 906 in order to control the spectrum output into room 900.

In at least some embodiments, light sources 901-904 may be positioned on different surfaces than those depicted. In at least some embodiments, light sources 901-904 may comprise different sizes and/or shapes. In at least some embodiments, light sources 901-904 may be used in addition to existing light sources unconnected with light sources 901-904 and/or light source controller 905. For example, light sources 901-904 may be used in addition to wall sconces or ceiling fixtures.

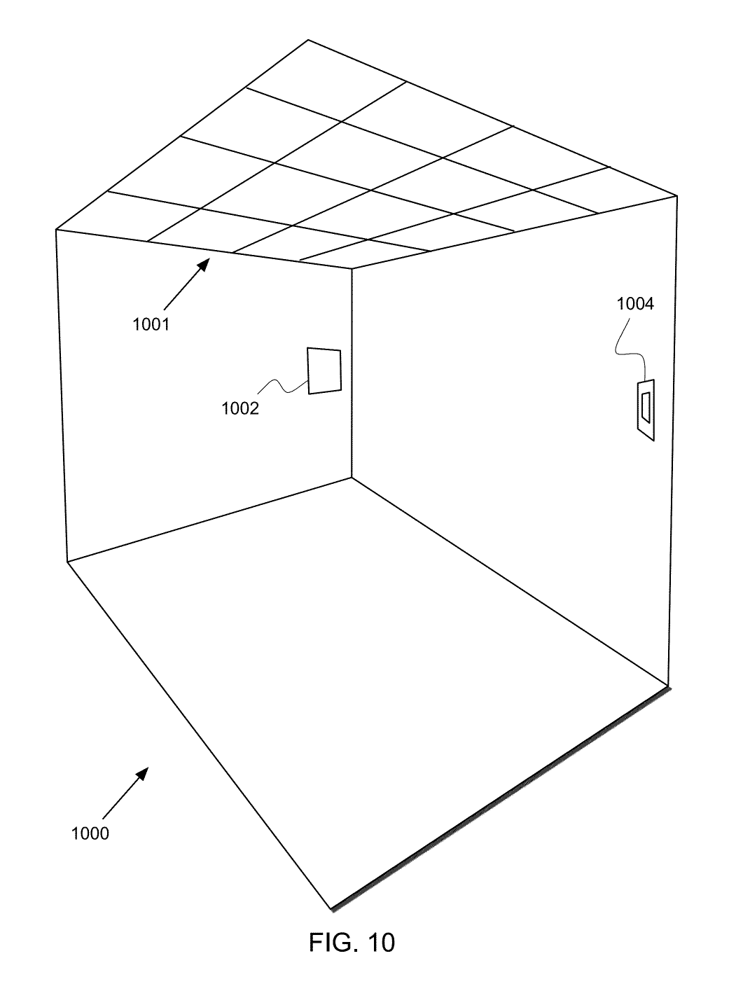

FIG. 10 depicts a perspective view of a room 1000 incorporating a lighting system according to another embodiment in which the room comprises a set of light sources 1001 configured as ceiling tiles. Similar to the lighting system described above with respect to room 900, the lighting system of room 1000 comprises a controller 1002 and a switch/control 1004 as described with respect to controller 905 and switch/control 906.

In at least some embodiments, light sources 1001 may be positioned on different surfaces than those depicted. In at least some embodiments, light sources 1001 may comprise different sizes and/or shapes. In at least some embodiments, light sources 1001 may be used in addition to existing light sources unconnected with light sources 1001 and/or light source controller 1002. For example, light sources 1001 may be used in addition to wall sconces or other ceiling light fixtures.

What is claimed is:

1. A fluorescent or light emitting diode-based system for generating light flux comprising:
a lamp including:
a plurality of light sources for illuminating an area, the light sources including a plurality of light-generating mechanisms at least one of which is different from the remainder, at least one of the light-generating mechanisms including at least one of a fluorescent light source or an LED light source, at least one of the plurality of light sources having a different color temperature output than another one of the plurality of light sources; and
a light source controller electrically coupled with the light sources and electrically coupled to a biological spectrum setting to control the color temperature output of the light sources by applying a percentage allocation of each of the light sources;
a power supply; and
a switch electrically coupled between the lamp and the power supply and arranged to control the supply of power from the power supply to the lamp.

2. The system for generating light flux of claim 1, wherein at least one of the plurality of light sources having a color temperature of less than 5,000 K.

3. The system for generating light flux of claim 1, wherein at least one of the plurality of light sources having a color temperature of greater than 5,000 K.

4. The system for generating light flux of claim 1, wherein at least one of the plurality of light sources having a color temperature of less than 5,000 K, and at least one of the plurality of light sources having a color temperature of greater than 5,000 K.

5. The system for generating light flux of claim 4, wherein the light source controller configured to apply a greater percentage allocation of the light source having a color temperature of less than 5,000 K than the light source having a color temperature of greater than 5,000 K.

6. The system for generating light flux of claim 4, wherein the light source controller configured to apply a greater percentage allocation of the light source having a color temperature of greater than 5,000 K than the light source having a color temperature of less than 5,000 K.

7. The system for generating light flux of claim 4,
wherein the light source controller configured to apply a greater percentage allocation of the light source having a color temperature of greater than 5,000 K than the light source having a color temperature of less than 5,000 K during a first period of time, and
wherein the light source controller configured to apply a greater percentage allocation of the light source having a color temperature of greater than 5,000 K than the light source having a color temperature of less than 5,000 K during a second period of time.

8. A method of providing light therapy to a human, comprising:
providing a lamp including a plurality of light sources for illuminating an area, the light sources including a plurality of light-generating mechanisms at least one of which is different from the remainder, at least one of the light-generating mechanisms including at least one of a fluorescent light source or an LED light source, at least one of the plurality of light sources having a different color temperature output than another one of the plurality of light sources; and
applying a percentage allocation to each of the plurality of light sources to control the color temperature output of the lamp for a period of time to affect a biological response in a human.

9. The method of claim 9, wherein the biological response in humans includes reduction of melatonin production, prevention of absorption of light by melanopsin, increase of the cortisol awakening response in humans, or suppression of cortisol levels in humans.

10. The method of claim 8, wherein at least one of the plurality of light sources having a color temperature of less than 5,000 K.

11. The method of claim 8, wherein at least one of the plurality of light sources having a color temperature of greater than 5,000 K.

12. The method of claim 8, wherein at least one of the plurality of light sources having a color temperature of less than 5,000 K, and at least one of the plurality of light sources having a color temperature of greater than 5,000 K.

13. The method of claim 12, further comprising applying a greater percentage allocation of the light source having a color temperature of less than 5,000 K than the light source having a color temperature of greater than 5,000 K.

14. The method of claim 12, further comprising applying a greater percentage allocation of the light source having a color temperature of greater than 5,000 K than the light source having a color temperature of less than 5,000 K.

15. A light fixture for generating light flux comprising:
a plurality of light sources for illuminating an area, the light sources including a plurality of light-generating mechanisms at least one of which is different from the remainder, at least one of the light-generating mechanisms comprising at least one of a fluorescent light source or an LED light source, at least one of the plurality of light sources having a different color temperature output than another one of the plurality of light sources;
a means for controlling a desired color temperature output of the light fixture;
a means for providing a desired biological spectrum to affect a biological response in a human.
a power supply; and
a switch electrically coupled between the lamp and the power supply and arranged to control the supply of power from the power supply to the plurality of light sources.

16. The light fixture of claim 15, further comprising a means for controlling the duration of time the light fixture outputs the desired color temperature.

17. The light fixture of claim 15, further comprising a means for controlling the desired color temperature output during a first period of time.

18. The light fixture of claim 17, further comprising a means for controlling the desired color temperature output during a second period of time.

19. The system for generating light flux of claim 15, wherein the desired color temperature is less than 5,000 K.

20. The system for generating light flux of claim 15, wherein the desired color temperature is greater than 5,000 K.
